(19) United States
(12) Patent Application Publication
(10) Pub. No.: US 2021/0145319 A1
(43) Pub. Date: May 20, 2021

(54) Method, apparatus, memory medium and terminal device for fall detection and protection

(71) Applicants: Chin Yau Tang, Hong Kong (HK); Nang Chong Yeung, Hong Kong (HK)

(72) Inventors: Chin Yau Tang, Hong Kong (HK); Nang Chong Yeung, Hong Kong (HK)

(21) Appl. No.: 17/094,802

(22) Filed: Nov. 10, 2020

(30) Foreign Application Priority Data
Nov. 18, 2019 (HK) 19132344.3

(51) Int. Cl. A61B 5/11; A41D 13/018; A61B 5/00

(52) U.S. Cl. CPC A61B 5/1117; A41D 13/018; A61B 5/7282; A61B 5/6804; A61B 5/4836

(57) ABSTRACT

Embodiments of the present invention disclose a method for fall detection and protection, which is configured to apply in a wearable device worn on a human body, the method comprising: obtaining a gesture variation information of chest or abdomen of the human body from the wearable device; determining whether the human body is in a dynamic state depending on the gesture variation information; if the human body is in a dynamic state, obtaining a gesture information of chest and abdomen of the human body at the present moment and determining if the human body is in a state of being about to fall; if the human body is in a state of being about to fall, determining whether the fall is an unconscious fall; and if the fall is an unconscious fall, protecting the human body. Embodiments of the present invention can accurately determine an unconscious fall and provide protection that can prevents injury.

### CROSS-REFERENCE TO RELATED APPLICATIONS

This application claims priority benefits to Hong Kong Patent Application No. 19132344.3, filed on Nov. 18, 2019. The contents of all of the aforementioned application are incorporated herein by reference.

### TECHNICAL FIELD

The present invention relates to the field of computer technology, particularly, a method, an apparatus, a memory medium and a terminal device for fall detection and protection.

### BACKGROUND ART

In everyday life, a prominent illness can cause people falling into a coma, for example, stroke caused by hypertension, cardiovascular diseases such as hypotension, hypoglycemia or myocardial infarction or so. For a heart disease, the prime time for emergency treatment is within 5 minutes after a heart attack, according to the World Health Organization, and for an ischemic stroke, the prime time for emergency treatment is within 3 hours. Every minute of delay reduces the survival chance by 10%, or may cause more severe aftereffects. If the patient suffers from such a prominent disease and is unsupported or unconscious, he or she will most probably fall to the ground. Falls may also cause accidental death for the elderly, the disabled, the chronically patents and for those, who require special care.

The problem to be solved is how to find out that the patient is in a state of being about to fall and to adopt certain measures to prevent the patient from falling, before the patient falls due to a prominent disease or an accident.

### DISCLOSURE OF INVENTION

The embodiments of the present invention provide a method, an apparatus, a memory medium and a terminal device for controlling of the digital output to solve or to ease one or technical problems in the prior art.

As one aspect of the embodiments of the present invention, the embodiments of the present invention provide a method for fall detection and protection configured to apply to a wearable device worn on human body, comprising: obtaining a gesture variation information of chest or abdomen of the human body from the wearable device; determining whether the human body is in a dynamic state depending on the gesture variation information; if the human body is in a dynamic state, obtaining a gesture information of chest and abdomen of the human body at the present moment and determining if the human body is in a state of being about to fall; if the human body is in a state of being about to fall, determining whether the fall is an unconscious fall; and if the fall is an unconscious fall, protecting the human body.

As one aspect of the embodiments of the present invention, the embodiments of the present invention provide a fall detection and protection apparatus configured to apply to a wearable device worn on human body, the apparatus comprising:

An information obtaining module, for obtaining from the wearable device a gesture variation information of chest and abdomen of the human body;

A dynamic state estimating module, for determining whether the human body is in a dynamic state depending on the gesture variation information;

A fall estimating module, for obtaining the gesture information of chest and abdomen of the human body at the present moment if the human body is in a dynamic state, and for determining whether the human body is in a state of being about to fall depending on the gesture information and gravity's pull;

A consciousness estimating module, for determining whether the fall is an unconscious fall depending on the gesture variation information, if the human body is in a state of being about to fall; and

A protection module, for protecting the human body if the fall is an unconscious fall.

As one aspect of the embodiments of the present invention, the embodiments of the present invention provide a wearable device, comprising: a wearable device body, for wearing on a human body; an airbag, which is provided in the wearable device body and comprises an inflator and a trigger device, which triggers the inflator to release noble gas, such that the wearable device body is filled and inflates; a processor, which is connected to the trigger device and implement the method provided in the previous embodiments, such that the trigger device is triggered when protection of the human body is undertaken.

As one aspect of the embodiments of the present invention, the embodiments of the present invention provide a design, in which a structure for fall detection and protection comprises a processor and a memory, wherein the memory is configured such that an apparatus for fall detection and protection implements an algorithm corresponding to the above described method for fall detection and protection, and wherein the processor is configured to implement an algorithm stored in the memory. The apparatus for fall detection and protection further comprises a communication interface, which is configured such that the apparatus for fall detection and protection communicates with other devices or communication network.

As one aspect of the embodiments of the present invention, the embodiments of the present invention provide a computer readable memory medium for computer software instructions of an apparatus for fall detection and protection, comprising an algorithm that is involved in implementation of the method for fall detection and protection.

By using the above described subject matter, the embodiments of the present invention can detect in a wearable device whether a human body falls and precisely estimate whether the fall of the human body happens consciously or unconsciously. Furthermore, it can provide protection during an unconscious fall of the human body effectively to avoid injury.

The above brief description is only intended to describe and not to limit the present invention by any means. In addition to the above described schematic aspects, embodiments and characteristics, further aspects, embodiments and characteristics of the present invention will be easily understood in conjunction with the figures and the detailed description below.

### BRIEF DESCRIPTION OF DRAWING

In the drawing, the use of the same reference symbols in different drawings indicates identical or similar items or elements unless otherwise noted.

The drawings are not necessarily to scale. It should be understood that the drawings only illustrate a few embodiments disclosed under the present invention and should not be regarded as limiting the scope of the present invention. In the drawings,

FIG. 1 shows a flow diagram of a method for fall detection and protection according to the present embodiments,

FIG. 2 shows a schematic view of a reference system according to the present embodiments when a human body stands,

FIG. 3 shows a schematic view of a reference system according to the present embodiments when a human body falls,

FIG. 4 shows a schematic view of a reference system according to the present embodiments,

FIG. 5 shows a structural schematic view of a fall detection and protection apparatus according to the present embodiments,

FIG. 6 shows a structural schematic view of a wearable device according to the present embodiments,

FIG. 7 shows a structural schematic view of a trigger device according to the present embodiments,

FIG. 8A shows a front view of a fall-proof clothing with an airbag before filling with noble gas according to the present embodiments,

FIG. 8B shows a side view of a fall-proof clothing with an airbag before filling with noble gas according to the present embodiments,

FIG. 8C shows a back view of a fall-proof clothing with an airbag before filling with noble gas according to the present embodiments,

FIG. 8D shows a front view of a fall-proof clothing with an airbag after filling with noble gas according to the present embodiments,

FIG. 8E shows a side view of a fall-proof clothing with an airbag after filling with noble gas according to the present embodiments,

FIG. 8F shows a back view of a fall-proof clothing with an airbag after filling with noble gas according to the present embodiments,

FIG. 9 shows a structural schematic view of a system for fall detection and protection according to the present embodiments,

FIG. 10 shows a structural schematic view of a terminal device according to the present embodiments.

### BEST MODE FOR CARRYING OUT THE INVENTION

Only some exemplary embodiments are briefly described below. It might be understood by those skilled in the art that the described embodiments can be modified by different ways without departing from the spirit or scope of the present invention. The drawings and the description are therefore considered to be exemplary in nature rather than limiting.

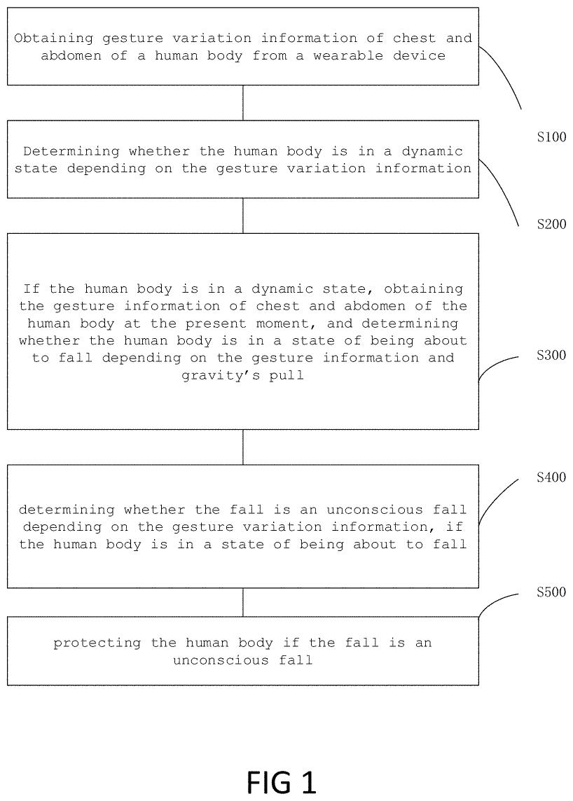

As one exemplary embodiment, FIG. 1 shows a flow diagram of a method for fall detection and protection configured to apply to a wearable device on a human body, e.g. a clothing with an airbag. During protection to a human body, the airbag is instantly filled and the clothing inflates. The method for fall detection and protection comprises step S100 and step S500, which are explained as follows:

S100, obtaining gesture variation information at chest and abdomen of a human body. When a wearable device is worn on a human body, at least the chest and abdomen of the human body are covered with sensors, such as accelerometer, gyroscope and etc. They can detect changes of position, velocity, acceleration, angles or so at the corresponding detection point. Gesture variation information may include information of changes of position, velocity, acceleration, angles or so. Chest may be one point or the central point in the chest are. Abdomen may be one point or the central point in the abdomen area. The central points are used as an example in the present embodiment.

In the present embodiment, gesture variation information may comprise change of position or a velocity parameter during the change in view of gesture of the human body within a defined time period, the velocity parameter comprises velocity, acceleration and angular velocity. A defined time period may be a period from a certain moment of the past to the present moment. In the present embodiment, a certain moment of the past may be determined by determining the time length. For example, by configuring a time length of 2 seconds, if time of the present moment is 18:15:30, then time of the past certain moment is 18:15:28.

S200, determining whether the human body is in a dynamic state depending on the gesture variation information.

In some embodiments, whether a human body is in a dynamic state can be determined depending on the gesture variations of two body parts (chest and abdomen) within a defined time period. If the gesture variations of chest and abdomen do not lie in a defined threshold value for gesture variations, it can be assumed that the human body is still in a static state, without estimating whether it is in a falling state. That is to say, there is no need to perform any subsequent estimating operations. However, once the gesture variations are detected to be dynamic in a further monitoring of the gesture variations of the human body, subsequent estimating operations can be performed.

For example, it is possible to determine whether the human body is in a static or dynamic state depending on variation amplitude, such as range, variance and standard deviation etc., of a certain parameter within a predefined time period. The parameters may comprise acceleration, angle, angular velocity etc.

In this embodiment, it is more accurate to estimate whether the human body is in a dynamic state depending on gesture variations at both chest and abdomen than to estimate whether the human body is in a dynamic state depending only on gesture variations at chest or at abdomen alone. When the human body is indeed in a static state, gesture at chest varies whereas gesture at abdomen not. If such estimation whether the human body is in a dynamic state is only based on the gesture variations at chest, it is possible that the human body is estimated to be in a dynamic state. Thus an estimation error occurs. Similarly, an estimation error may also likely occur, if estimation whether the human body is in a dynamic state is only based on the gesture variations at abdomen.

S300, if the human body is in a dynamic state, obtaining the gesture information of chest and abdomen of the human body at the present moment, and determining whether the human body is in a state of being about to fall depending on the gesture information and gravity's pull.

For example, in a defined coordinate direction, the current gesture information may comprise acceleration, velocity, angular velocity, position and angle etc. The deviation angle of chest and abdomen of the human body relative to gravity's pull and then whether the human body is in a state of being about to fall is determined by gesture information and the gravity's pull. An about-to-fall state may be that the person is falling but has not landed.

S400, determining whether the fall is an unconscious fall depending on the gesture variation information, if the human body is in a state of being about to fall.

In some embodiments, a gesture variation information may comprise variation data of multiple parameters. Whether a fall is estimated to be a conscious fall or an unconscious fall is based on all data of the multiple parameters in the gesture variation information. If the multiple parameters satisfy the predefined conditions at the same time, the fall is assumed to be an unconscious fall.

In some embodiments, whether a fall is estimated to be a conscious fall or an unconscious fall is based on some data of the multiple parameters in the gesture variation information. For example, one datum is selected from all variation data of the multiple parameters to determine whether the fall is an unconscious fall. For example, a median, a range or a maximum etc. is selected.

S500, protecting the human body if the fall is an unconscious fall.

In some embodiments, protective operations of the human body may comprise but not limited to the following: in case that the wearable device is clothing with a gas pocket, the airbag is instantly filled and the clothing inflates to prevent people from being injured when they fall to the floor or the ground; sending alarm information; sending messages or phone calls to contacts.

For example, a gesture variation information may comprise change in acceleration and change in angular velocity within the predefined time period, such as change in acceleration and change in velocity within a time period from the past to the present two seconds, change in acceleration and change in velocity within a time period from the past to the present one second etc.

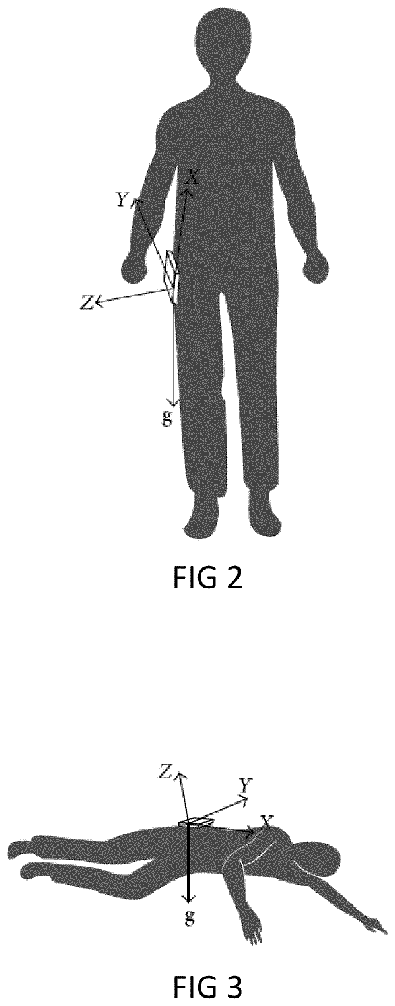

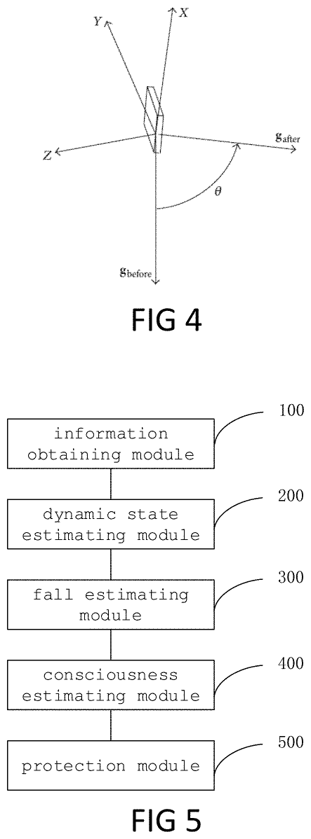

Each of FIGS. 2 and 3 shows a three-dimensional reference coordinate system when people stand or when people fall, see FIGS. 2 to 4. FIG. 4 shows a three-dimensional reference coordinate system of the present embodiments. Suppose a human body is a cube, the X-axis refers to a length direction of the human torso, the Y-axis references a direction perpendicular to the human torso and to the gravity's pull and the Z-axis lies in the same plane as the Y-axis and is perpendicular to both the X- and Y-axes.

The acceleration of the chest can be expressed as:

a_{A}={square root over (a_{A}_{ₓ}²+a_{A}_{_{y}}²+a_{A}_{_{z}}²)}

The acceleration of the abdomen can be expressed as:

a_{B}={square root over (a_{B}_{ₓ}²+a_{B}_{_{y}}²+a_{B}_{_{z}}²)}

The angular velocity of the chest can be expressed as:

ω_{A}={square root over (ω_{A}_{ₓ}²+ω_{A}_{_{y}}²+ω_{A}_{_{z}}²)}

The angular velocity of the abdomen can be expressed as:

ω_{B}={square root over (ω_{B}_{ₓ}²+ω_{B}_{_{y}}²+ω_{B}_{_{z}}²)}

wherein a_{A}_{ₓ} is the acceleration of the chest on the X-axis, a_{A}_{_{y}} is the acceleration of the chest on the Y-axis, a_{A}_{_{z}} is the acceleration of the chest on the Z-axis; a_{B}_{ₓ} is the acceleration of the abdomen on the X-axis, a_{B}_{_{y}} is the acceleration of the abdomen on the Y-axis, a_{B}_{_{z}} is the acceleration of the abdomen on the Z-axis; ω_{A}_{ₓ} is the angular velocity of the chest on the X-axis, ω_{A}_{_{y}} is the angular velocity of the chest on the Y-axis, ω_{A}_{_{z}} is the angular velocity of the chest on the Z-axis; ω_{B}_{ₓ} is the angular velocity of the abdomen on the X-axis, ω_{B}_{_{y}} is the angular velocity of the abdomen on the Y-axis, ω_{B}_{_{z}} is the angular velocity of the chest on the Z-axis.

For example, in the above described step S400, estimating whether the fall of the human body is an unconscious fall may further comprise:

firstly, obtaining from the gesture variation information an acceleration maximum and an angular velocity maximum of the chest as well as an acceleration maximum and an angular velocity maximum of the abdomen;

then determining whether the fall is an unconscious fall depending on the acceleration maximum and the angular velocity maximum of the chest and the acceleration maximum and the angular velocity maximum of the abdomen;

wherein the acceleration maximum of the chest can be expressed as a_{A}_{ₘₐₓ}, the acceleration maximum of the abdomen can be expressed as a_{B}_{ₘₐₓ}, the angle acceleration maximum of the chest can be expressed as ω_{A}_{ₘₐₓ} and the angle acceleration maximum of the abdomen can be expressed ω_{B}_{ₘₐₓ}.

In some embodiments, the above determination process can be performed by providing some thresholds. If the acceleration maximum and the angular velocity maximum are both greater than the defined thresholds, it shows that the fall is an unconscious fall. If some values are greater than the thresholds and the others not, it proves that the human body is aware that it is in a state of being about to fall. Since people are aware that they are in a state of being about to fall, corresponding reactions are undertaken, such as holding on to the surrounding obstacles, which bends their chest slightly but still keeps the people in a standing or sitting position, so that some values do not exceed the defined thresholds. For example, the data of the chest is greater than the defined thresholds and the data of the abdomen not.

For example, if the acceleration maximum of the chest is greater than a first acceleration threshold, the angular velocity maximum of the chest is greater than a first angular velocity threshold, the acceleration maximum of the abdomen is greater than a second acceleration threshold and the angular velocity maximum of the abdomen is greater than the second angular velocity threshold, a fall can be determined as an unconscious fall.

In some embodiments, since the acceleration of the chest changes more during an unconscious fall than the acceleration of the abdomen and the angular velocity of the chest changes less than the angular velocity of the abdomen, the first acceleration threshold can be defined to be greater than the second acceleration threshold and the first angular velocity threshold less than the second angular velocity threshold. For example, taking the gravity g as a reference, the first acceleration threshold can be 3.0 g, the second acceleration threshold 2.5 g, the first angular velocity threshold 200°/s and the second angular velocity threshold 340°/s.

For example, in the above described step S200, if the gestures variations of both chest and abdomen are detected to exceed the defined range, it proves that the human body is in a dynamic state, e.g. bending the body, changing from sitting or lying to standing, or changing from standing to sitting or lying. Determining whether the human body is in a dynamic state depending on the gesture variation information may further comprise:

firstly, determining the acceleration range and the angular velocity range of the chest and the acceleration range and the angular velocity range of the abdomen;

then if the acceleration range of the chest is less than a first acceleration range threshold, the acceleration range of the abdomen is less than a second acceleration range threshold, the angular velocity range of the chest is less than a first angular velocity range threshold and the angular velocity range of the abdomen is less than a second angular velocity range threshold, the human body is determined to be in a dynamic state.

In this embodiment, the first acceleration range threshold can be the same as the second acceleration range threshold and the first angular velocity range threshold can be the same as the second angular velocity range threshold.

For example, if the gesture variation information satisfies the following expression 1, the human body can be assumed to be in a dynamic state:

$$\vert {a}_{{A}_{max}} - {a}_{{A}_{min}} \vert < 0.4 g ⋂ \vert {a}_{{B}_{max}} - {a}_{{B}_{min}} \vert < 0.4 g ⋂ \vert {ω}_{{A}_{max}} - {ω}_{ {A}_{min}} \vert < {60}^{∘} / s ⋂ \vert {ω}_{{B}_{max}} - {ω}_{{B}_{min}} \vert < {60}^{∘} / s \qquad ( 1 )$$

In other embodiments, the human body may be determined to be in a dynamic state by determining the acceleration range and the angular velocity range of the chest and the acceleration range and the angular velocity range of the abdomen. The human body may also be determined to be in a dynamic state by determining the acceleration standard deviation and the angular velocity standard deviation of the chest and the acceleration standard deviation and the angular velocity standard deviation of the abdomen.

After that the gesture variation information satisfies the expression 1 and after determining that the human body is in a dynamic state, the acceleration of the human body at the present moment in the length direction of the human torso and the acceleration of the abdomen in the length direction of the human torso are obtained, see FIGS. 2 to 4. The angle variation of the human torso in the length direction relative to the gravity pull's direction can be determined through the obtained data when considering the chest as the origin, and the angle variation of the human torso in the length direction relative to the gravity pull's direction when considering the abdomen as the origin. Depending on these two angle variations, state of the human body can be estimated, such as standing, bending, sitting or lying etc. If the human body is estimated to be about to lie down, that means the person is in a state of being about to fall.

For example, in the above described step S300, determining whether the human body is in a state of being about to fall depending on the gesture information and the gravity's pull, may further comprise:

firstly, determining the angle of the chest between the length direction along the human torso and the direction of the gravity's pull depending on the acceleration of the chest in the length direction of the human torso and the gravity's pull,

wherein in some embodiments, this angle can be calculated according to the following expression 2:

$${θ}_{A} = arccos \frac{a {A}_{x}}{g} \qquad ( 2 )$$

wherein θ_{A} is an angle of the chest between the length direction along the human torso and the direction of the gravity's pull, aAₓ is the acceleration of the chest along the length direction of the human torso and g is the gravity's pull;

Secondly, determining the angle of the abdomen between the length direction along the human torso and the direction of the gravity's pull depending on the acceleration of the abdomen in the length direction of the human torso and the gravity's pull,

wherein in some embodiments, this angle can be calculated according to the following expression 3:

$${θ}_{B} = arccos \frac{a {B}_{x}}{g} \qquad ( 3 )$$

wherein θ_{B} is the angle of the abdomen between the length direction of the human torso and the direction of the gravity's pull, aBₓ is the acceleration of the abdomen in the length direction of the human torso and g is the gravity's pull.

In some embodiments, estimating of a gesture can refer to the following table 1:

TABLE 1
relationship between angles and gestures

| Gestures | θ_{A}(°) | θ_{B}(°) |
|---|---|---|
| Standing | <35 | <35 |
| Bending | >35 | <35 |
| Sitting | <35 | >35 |
| Lying | >35 | >35 |

In conjunction with table 1, if θ_{A} of the chest of the human body at the present moment is greater than 35° and θ_{B} of the abdomen of the human body is greater than 35°, it can be determined that the human body is in a state of being about to fall down to the ground and a further estimation is required to protect the human body.

In the present embodiments, having determined that the human body is in a state of being about to fall, a further estimating, in which whether the human body falls unconsciously or consciously is estimated, prevents waste of the protection measures and improves resource utilization rate. For example, in case a conscious fall of the human body is estimated, it will not make much difference if the wearable device is now filled and inflates, since the person will take corresponding actions, such as holding on to the surrounding obstacles, to stand up.

As an exemplary embodiment, FIG. 5 shows a fall detection and protection apparatus provided by the embodiments of the present invention configured to apply to a wearable device worn on human body, the apparatus can comprise:

an information obtaining module 100, for obtaining from the wearable device a gesture variation information of chest and abdomen of the human body;

a dynamic state estimating module 200, for determining whether the human body is in a dynamic state depending on the gesture variation information;

a fall estimating module 300, for obtaining the gesture information of chest and abdomen of the human body at the present moment if the human body is in a dynamic state, and for determining whether the human body is in a state of being about to fall depending on the gesture information and gravity's pull;

a consciousness estimating module 400, for determining whether the fall is an unconscious fall depending on the gesture variation information, if the human body is in a state of being about to fall; and

a protection module 500, for protecting the human body if the fall is an unconscious fall.

In some embodiments, the gesture variation information comprises an acceleration variation and an angular velocity variation within a predefined time period.

In some embodiments, the consciousness estimating module 400 comprises:

a maximum obtaining unit, for obtaining an acceleration maximum and an angular velocity maximum of the chest and an acceleration maximum and an angular velocity maximum of the abdomen; and

an estimating unit, for determining whether the fall is an unconscious fall depending on the acceleration maximum and the angular velocity maximum of the chest and the acceleration maximum and the angular velocity maximum of the abdomen.

In some embodiments, the estimating unit apples specifically in that: if the acceleration maximum of the chest is greater than a first acceleration threshold, the angular velocity maximum of the chest is greater than a first angular velocity threshold, the acceleration maximum of the abdomen is greater than a second acceleration threshold and the angular velocity maximum of the abdomen is greater than a second angular velocity threshold, the fall is an unconscious fall.

In some embodiments, the first acceleration threshold is greater than the second acceleration threshold and the first angular velocity threshold is less than the second angular velocity threshold.

In some embodiments, the dynamic state estimating module 200 may comprise:

a range determining unit, for determining an acceleration range and an angular velocity range of the chest and an acceleration range and an angular velocity range of the abdomen;

a dynamic state estimating unit, for determining that the human body is in a dynamic state, if the acceleration range of the chest is less than a first acceleration range threshold, the acceleration range of the abdomen is less than a second acceleration range threshold, the angular velocity range of the chest is less than a first angular velocity range threshold and the angular velocity range of the abdomen is less than a second angular velocity range threshold.

In some embodiments, the gesture information comprise an acceleration of the chest in the length direction of the torso and an acceleration of the abdomen in the length direction of the torso, and the fall estimating module 300 comprises:

a first angle determining unit, for determining an angle of the chest between the length direction of the torso and the direction of the gravity's pull depending on the acceleration of the chest in the length direction of the torso and the gravity's pull;

a second angle determining unit, for determining an angle of the abdomen between the length direction of the torso and the direction of the gravity's pull depending on the acceleration of the abdomen in the length direction of the torso and the gravity's pull.

In some embodiments, the first angle determining unit follows the following expression:

$${θ}_{A} = arccos \frac{a {A}_{x}}{g}$$

wherein θ_{A} is an angle of the chest between the length direction along the human torso and the direction of the gravity's pull, aAₓ is the acceleration of the chest along the length direction of the human torso and g is the gravity's pull.

In some embodiments, the second angle determining unit follows the following expression:

$${θ}_{B} = arccos \frac{a {B}_{x}}{g}$$

wherein θ_{B} is the angle of the abdomen between the length direction of the human torso and the direction of the gravity's pull, aBₓ is the acceleration of the abdomen in the length direction of the human torso and g is the gravity's pull.

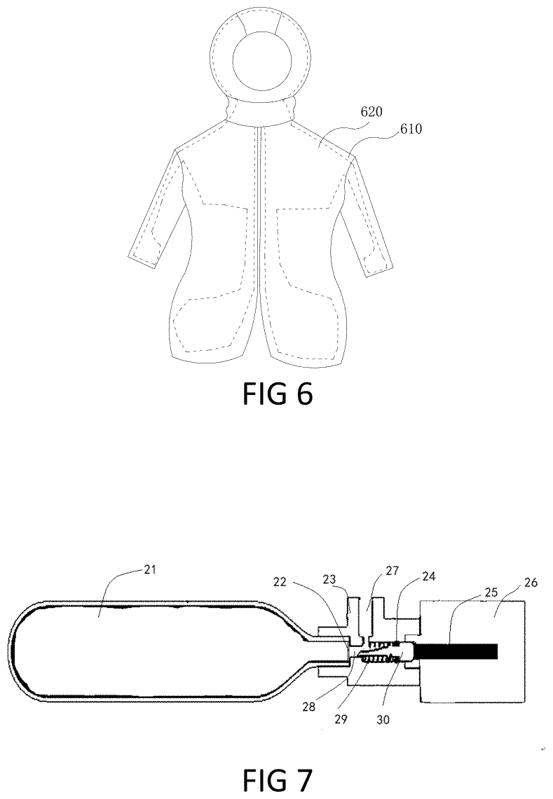

As an exemplary embodiments, FIG. 6 shows a wearable device, comprising:

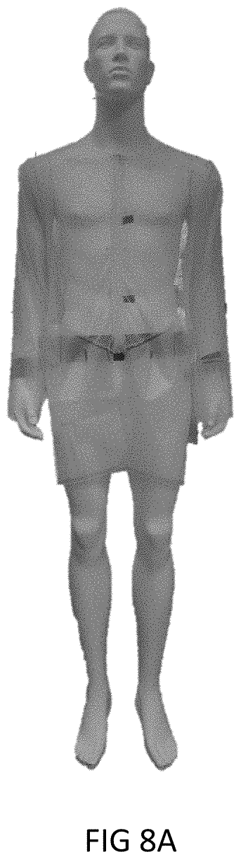

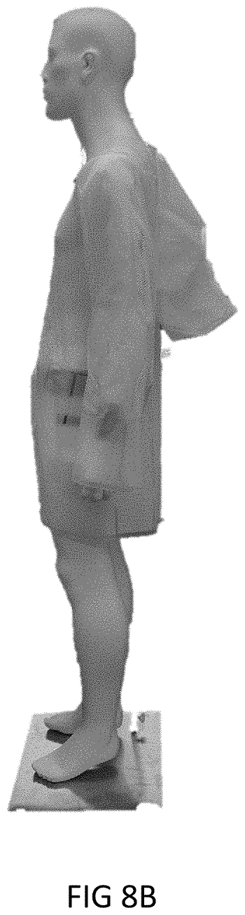

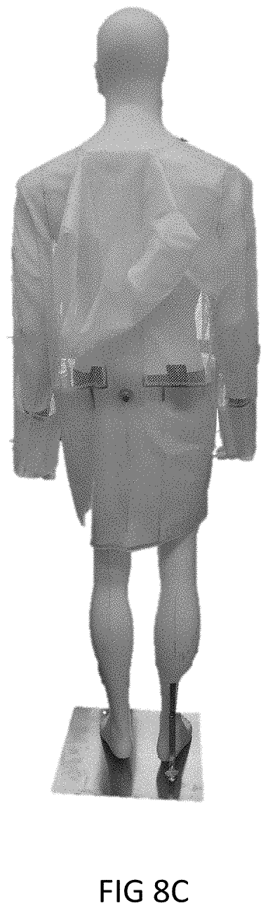

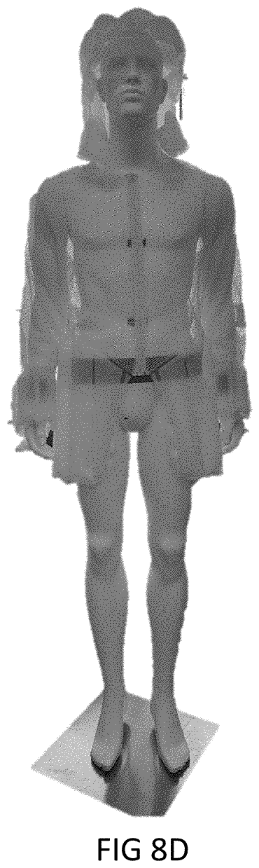

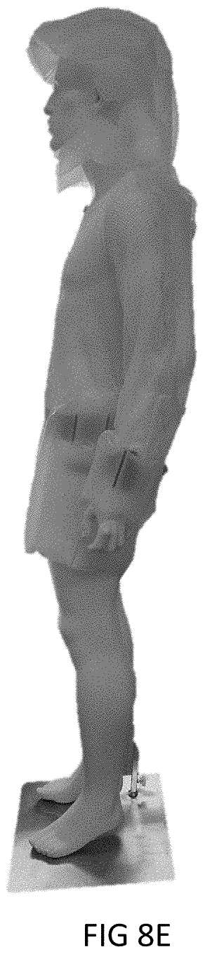

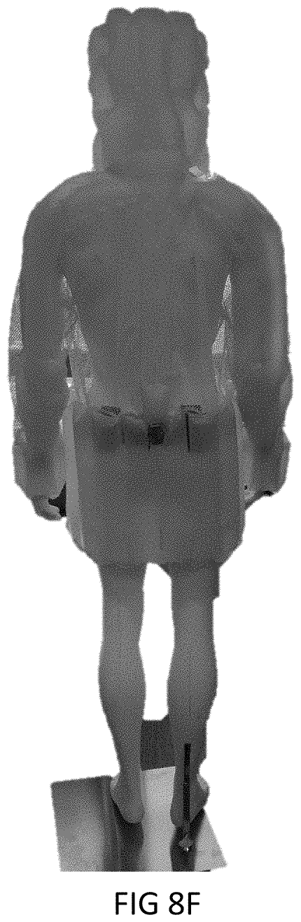

a wearable device body 610, for wearing on a human body. As shown in FIG. 8A, the wearable device can be a top, which is worn on a mannequin;

an airbag 620, which is provided in the wearable device body and comprises an inflator (not shown in FIG. 6) and a trigger device, which triggers the inflator to release gas, such that the wearable device body is filled and inflates (not shown in FIG. 6). The airbag 620 can be formed in one or multiple parts, for example, distributed on hat, shoulder, sleeve and positions that near waist and hip of the clothing;

a processor (not shown), which is connected to the trigger device and implement the method provided in any one of the previous embodiments, such that the trigger device is triggered when protection of the human body is undertaken, wherein the processor can be provided inside the wearable device body. Besides implementing the method provided in any one of the previous embodiments, the processor can be equipped with a GPS system, which allows a real-time tracking of the people who fell. The processor can also be considered as a control unit.

In addition, the wearable device can be provided with a few sensors at the positions of the wearable device body 610 that is near to the human chest and abdomen, such as acceleration sensors, angular velocity sensors and gyroscopes etc.

In some embodiments, an inflator can be used to fill the airbag.

In some embodiments, the processor is equipped with a communication module, for connecting with a cloud data server, wherein the cloud data server informs the emergency contact or the nearest medical center and sends a request of emergency assistance.

In some embodiments, the person himself, family or medical personnel, who wears the above described wearable device, can access the data relating to himself through a web portal or a mobile app. Data such as gesture variation information detected by the above described wearable device, estimating process and results etc. can be uploaded to the cloud data server through the communication module and be saved for data access.

For example, FIG. 7 shows a structure of a trigger device provided in the present embodiments, wherein the inflator 21 is covered at its opening with a sealing foil 22, which can be made of plastic or thin foils to seal the opening of the inflator 21 and is easily punctured by a sharp object. The trigger device can comprise a solenoid valve unit 26, a plunger 30 and a connector 23 that is equipped with interconnected vent pipes 27 and a plunger channel 28. The connector 23 has two ends at the plunger channel 28 and is connected between the opening of the inflator 21 and the solenoid valve unit 26, wherein a first end of the plunger channel 28 aligns with the opening of the inflator 21. In addition, a gas inlet of the vent pipes 27 is positioned near to the first end of the plunger channel 28, such that noble gas can escape from the opening of the inflator 21, pass the first end of the plunger channel 28 and enter the gas inlet of the vent pipes, when the inflator 21 is punctured.

A sharp tail of the plunger 30 is inserted into the plunger channel 28 from a second end of the plunger channel 28 and is positioned inside the plunger channel 28. Head of the plunger 30 is attached to the second end of the plunger channel 28 and the tail is at a certain distance from the first end of the plunger channel 28, i.e. the opening of the inflator 21, and the maximum radius of the cross-section of the plunger 30 is no greater than the cross-section radius of the plunger channel 28.

The solenoid valve unit 26 is provided with a magnetic bar 25, which can be in contact with or close to the head of the plunger 30 such that when the trigger device is triggered, the magnetic bar 25 collides with the head of the plunger 30, the impact moves the plunger 30 forwards inside the plunger channel 28 and the sharp tail of the plunger 30 punctures the sealing foil 22 at the opening of the inflator 21, so that the noble gas inside the inflator 21 escapes to the airbag of the wearable device body through the vent pipes 27.

In the present embodiment, as shown in FIG. 7, the plunger channel 28 is connected with the vent pipes 27 and they are perpendicular to each other. Since the plunger 30 is a large-ended structure, the gas inlet of the vent pipes 27 is located at its first end which is close to the plunger channel 28, such that even if the plunger 30 moves forward to puncture the inflator 21, the plunger 30 will not block the gas path of the vent pipes 27 and the plunger channel 28.

In some embodiments, the plunger channel 28 is equipped with a screw thread 29 to avoid that the plunger 30 moves and punctures the sealing foil 22 before the trigger device is triggered. The screw thread 29 is easy-to-fray or soft. When the plunger 30 is hit by the magnetic bar 25, the plunger 30 can break through the screw thread 29 and move forwards until the first end of the plunger channel 28.

In some embodiments, the plunger channel 28 is equipped with a sealing ring 24, which is located between the head of the plunger 30 and the plunger channel 28, such that it can be avoided, that noble gas of the inflator 21 escapes from the second end of the plunger channel 28 into the solenoid valve unit 26 after the sealing foil 22 is punctured.

FIG. 8A to 8F show a front view, a side view and a back view of the wearable device before and after filling with the noble gas. When a patient falls accidentally and is determined by algorithms that the fall is an unconscious fall, the trigger device will be triggered and then punctures the high pressure inflator. Noble gas is released to fill the airbag of the clothing worn on the patient body. The airbag can spread out within milliseconds to protect body parts of the patients which may be injured easily, such as head, arms and wrists, such that the injury level and death rate caused by falls are reduced.

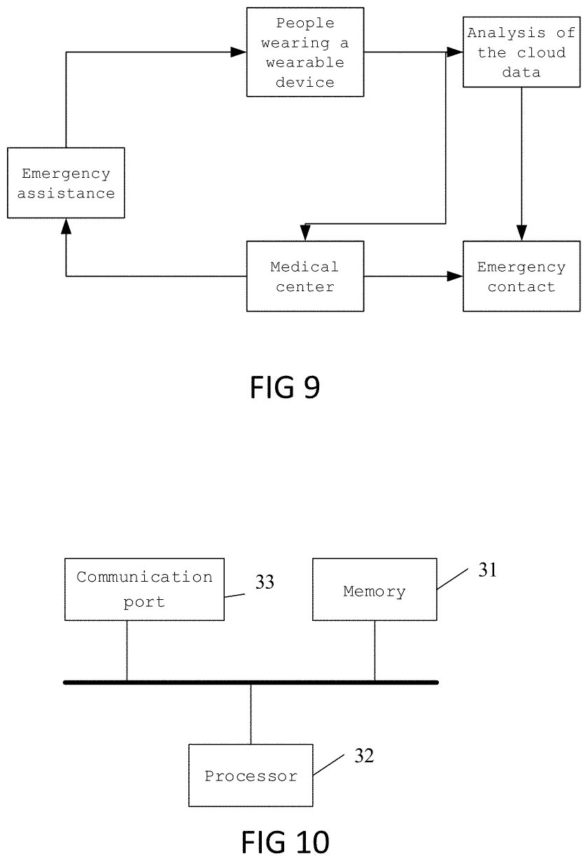

See FIG. 9, in the event that a patient is found fell or in coma, a satellite navigation system tracks the patient's location in real time, and the cloud data server sends alerts simultaneously to inform the family/medical center for emergency assistance. Under normal circumstances, patients, family members and medical personal can access the relevant data in real time through online platforms and mobile applications.

The functions of the apparatus can be realized by hardware or by hardware execution of the corresponding software. The hardware or software comprises one or more modules corresponding to the above described functions.

As one example of the present embodiments, the present embodiments provide a design, in which a processor and a memory are included in a fall detection and protection structure. The memory is configured to execute algorithms that correspond to the above described methods for fall detection and protection. The fall detection and protection apparatus further comprises a communication port configured to communicate the fall detection and protection apparatus with other devices or communication network.

The device further comprises:

a communication port 33, for communication between the processor 32 and the extern devices.

The memory 31 may comprise high speed RAM memory, but it may also comprise a non-volatile memory, such as at least one disk memory.

If the processor 31, the processor 32 and the communication port 33 are realized separately, the processor 31, the processor 32 and the communication port 33 can connect and communicate with each other through the bus. The bus can be an ISA (Industry Standard Architecture) bus, a PCI (peripheral component) bus or an EISA (extended industry standard component) bus etc. The bus can be grouped into address bus, data bus, control bus etc. In convenience of presentation, FIG. 10 shows only one thick line to represent the bus. It does not mean that only one bus or bus of only one kind is provided.

Optionally, if the processor 31, the processor 32 and the communication port 33 are integrated in a single chip in practice, the processor 31, the processor 32 and the communication port 33 can communicate with each other through internal ports.

Reference in the description to the terms like “an embodiment”, “some embodiments”, “an example”, “a particular example” or “some examples” means that a particular feature, structure, material or characteristic described in connection with the embodiment or example is included in at least one embodiment or example of the present invention. Furthermore, the particular feature, structure, material or characteristic may be combined in any suitable manner in one or more embodiments. In addition, those skilled in the art can combine or group different embodiments or examples or different features of the embodiments or examples described in this description, unless they contradict another.

In addition, terms such as “first” and “second” are used herein for purposes of description and are not intended to indicate or imply relative importance or significance. Thus, the feature defined with “first” and “second” may include one or more this feature. In the description of the present disclosure, “plurality” is defined as a number of two or more than two, unless specified otherwise.

It should be understandable for those skilled in the art that, the flow chart or any process or method described herein in other manners may represent a module, segment, or portion of code that includes one or more executable instructions to implement the specified logic function(s) or that includes one or more executable instructions of the steps of the progress. Although the flow chart shows a specific order of execution, it is understood that the order of execution may differ from that which is depicted, including execution in a simultaneous or reverse order according to the referred function(s).

The logic and step described in the flow chart or in other manners, for example, a scheduling list of an executable instruction to implement the specified logic function(s), it can be embodied in any computer-readable medium for use by or in connection with an instruction execution system such as, for example, a processor in a computer system or other system. In this sense, the logic may include, for example, statements including instructions and declarations that can be fetched from the computer-readable medium and executed by the instruction execution system. In the context of the present disclosure, a “computer-readable medium” can be any medium that can contain, store, or maintain the printer registrar for use by or in connection with the instruction execution system.

The computer-readable medium may be a computer-readable signal medium or a computer-readable storage medium or the combination thereof. Examples of computer-readable storage media (non-exhaustive list) include: electrical connections with one or more wires, portable computer disks, hard disk RAM, read-only memory (Read-Only Memory, ROM), erasable programmable Read-only memory (Erasable Programmable Read-Only Memory, EPROM) or flash memory, optical fiber, portable CD-ROM, optical storage device, magnetic storage device, or any suitable combination of the foregoing. In this document, the computer-readable storage medium can be any tangible medium that contains or stores an algorithm, and the algorithm can be used by or in combination with an instruction execution system, apparatus, or device.

The computer-readable signal medium may include a data signal propagated in baseband or as a part of a carrier wave, and the computer-readable signal medium carries computer-readable algorithm code. This propagated data signal can take many forms, including but not limited to electromagnetic signals, optical signals, or any suitable combination of the foregoing. The computer-readable signal medium may also be any computer-readable medium other than the computer-readable storage medium. The computer-readable medium may send, propagate, or transmit the algorithm for use by or in combination with the instruction execution system, apparatus, or device. The algorithm code contained on the computer-readable medium can be transmitted by any suitable medium, including but not limited to wireless, wire, optical cable, radio frequency (RF), etc., or any suitable combination of the foregoing.

Those skilled in the art will recognize that these embodiments may be implemented in hardware, software, firmware, or any combination thereof. In one or more example embodiments, the functions and methods described may be implemented in hardware, software, or firmware executed on a processor, or any combination thereof. For example, In hardware embodiments, as in another embodiment, the systems may be implemented with any or a combination of the following, or other, technologies, which are all well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

Ordinary people skilled in the art may understand that all or part of the steps carried out to implement the above described embodiments may be realized by an algorithm that instructs the relevant hardware, the algorithm can be stored in a computer-readable storage medium. When the algorithm is executed, one of the steps according to the method embodiments or the combination thereof are included.

In addition, each functional unit in each embodiment of the present invention may be integrated into one processing module, or each unit may exist alone physically, or two or more units may be integrated into one module. The integrated modules can be implemented in form of hardware or software function modules. If the integrated module is implemented in form of a software functional module and sold or used as an independent product, it may also be stored in a computer-readable storage medium. The storage medium may be a read-only memory, a magnetic disk or an optical disk.

It should be appreciated that the foregoing is only preferred embodiments of the present invention and is not intended to limit the protection scope of the present invention. It is apparent for those skilled in the art to make various modifications and variations to the present invention within the revealed technical field of the present invention. The present invention is intended to cover these modifications and variations provided that they fall in the protection scope defined in the present invention. Thus, claims define the protection scope of the present invention, for which protection is sought.

We claim:

1. A method for fall detection and protection, characterized in that the method is configured to apply to a wearable device worn on a human body, the method comprising:
obtaining a gesture variation information of chest or abdomen of the human body from the wearable device;
determining whether the human body is in a dynamic state depending on the gesture variation information;
if the human body is in a dynamic state, obtaining a gesture information of chest and abdomen of the human body at the present moment and determining if the human body is in a state of being about to fall;
if the human body is in a state of being about to fall, determining whether the fall is an unconscious fall; and
if the fall is an unconscious fall, protecting the human body.

2. The method as claimed in claim 1, characterized in that the gesture variation information comprises an acceleration change and an angular velocity change within a predefined time period.

3. The method as claimed in claim 2, characterized in that determination whether the fall is an unconscious fall depending on the gesture variation information comprises:
Obtaining from the gesture variation information an acceleration maximum and an angular velocity maximum of the chest as well as an acceleration maximum and an angular velocity maximum of the abdomen;
determining whether the fall is an unconscious fall depending on the acceleration maximum and the angular velocity maximum of the chest as well as the acceleration maximum and the angular velocity maximum of the abdomen.

4. The method as claimed in claim 3, characterized in that determination whether the fall is an unconscious fall comprises:
if the acceleration maximum of the chest is greater than a first acceleration threshold, the angular velocity maximum of the chest is greater than a first angular velocity threshold, the acceleration maximum of the abdomen is greater than a second acceleration threshold and the angular velocity maximum of the abdomen is greater than the second angular velocity threshold, the fall is an unconscious fall.

5. The method as claimed in claim 4, characterized in that the first acceleration threshold is greater than the second acceleration threshold and the first angular velocity threshold is less than the second angular velocity threshold.

6. The method as claimed in claim 2, characterized in that determination whether the human body is in a dynamic state depending on the gesture variation information comprises:
determining an acceleration range and an angular velocity range of the chest as well as an acceleration range and an angular velocity range of the abdomen;
if the acceleration range of the chest is less than a first acceleration range threshold, the acceleration range of the abdomen is less than a second acceleration range threshold, the angular velocity range of the chest is less than a first angular velocity range threshold and the angular velocity range of the abdomen is less than a second angular velocity range threshold, the human body is determined to be in a dynamic state.

7. The method as claimed in claim 2, characterized in that the gesture information comprise an acceleration of the chest in the length direction of the torso and an acceleration of the abdomen in the length direction of the torso and in that determination whether the human body is in a state of being about to fall depending on the gesture information and the gravity's pull comprises:
determining an angle of the chest between the length direction of the torso and the direction of the gravity's pull depending on the acceleration of the chest in the length direction of the torso and the gravity's pull;
determining an angle of the abdomen between the length direction of the torso and the direction of the gravity's pull depending on the acceleration of the abdomen in the length direction of the torso and the gravity's pull.

8. The method as claimed in claim 7, characterized in that determination of an angle of the chest between the length direction of the torso and the direction of the gravity's pull depending on the acceleration of the chest in the length direction of the torso and the gravity's pull comprises the following expression:
$${θ}_{A} = arccos \frac{a {A}_{x}}{g}$$
wherein θ_{A} is an angle of the chest between the length direction along the human torso and the direction of the gravity's pull, aAₓ is the acceleration of the chest along the length direction of the human torso and g is the gravity's pull.

9. The method as claimed in claim 7, characterized in that determination of an angle of the abdomen between the length direction of the torso and the direction of the gravity's pull depending on the acceleration of the abdomen in the length direction of the torso and the gravity's pull comprises the following expression:
$${θ}_{B} = arccos \frac{a {B}_{x}}{g}$$
wherein θ_{B} is the angle of the abdomen between the length direction of the human torso and the direction of the gravity's pull, aBₓ is the acceleration of the abdomen in the length direction of the human torso and g is the gravity's pull.

10. A fall detection and protection apparatus, characterized in that the apparatus is configured to apply to a wearable device worn on human body, the apparatus comprising:
an information obtaining module, for obtaining from the wearable device a gesture variation information of chest and abdomen of the human body;
a dynamic state estimating module, for determining whether the human body is in a dynamic state depending on the gesture variation information;
a fall estimating module, for obtaining the gesture information of chest and abdomen of the human body at the present moment if the human body is in a dynamic state, and for determining whether the human body is in a state of being about to fall depending on the gesture information and gravity's pull;
a consciousness estimating module, for determining whether the fall is an unconscious fall depending on the gesture variation information, if the human body is in a state of being about to fall; and
a protection module, for protecting the human body if the fall is an unconscious fall.

11. A wearable device, characterized in that the wearable device comprises:
a wearable device body, for wearing on a human body;
an airbag, which is provided in the wearable device body and comprises an inflator and a trigger device, which triggers the inflator to release gas, such that the wearable device body is filled and inflates;
a processor, which is connected to the trigger device and implement any one of the methods as claimed in claims 1, such that the trigger device is triggered when undertaking protection of the human body.

12. The wearable device as claimed in claim 11, characterized in that the inflator is covered at its opening with a sealing foil, wherein the trigger device comprises a solenoid valve unit, a plunger and a connector that is equipped with interconnected vent pipes and a plunger channel; wherein the connector is connected between the opening of the inflator and the solenoid valve unit, and a first end of the plunger channel aligns with the opening of the inflator; wherein a sharp tail of the plunger is inserted into a second end of the plunger channel and is positioned inside the plunger channel, wherein a head of the plunger is attached to the second end of the plunger channel and the tail is positioned at a certain distance from the first end of the plunger channel, and the maximum radius of the cross-section of the plunger is no greater than the cross-section radius of the plunger channel; wherein
the solenoid valve unit is provided with a magnetic bar such that when the trigger device is triggered, the magnetic bar collides with the head of the plunger and the impact moves the plunger forwards inside the plunger channel and the sharp tail of the plunger punctures the sealing foil at the opening of the inflator, so that the noble gas inside the inflator escapes to the airbag of the wearable device body through the vent pipes.

13. The device as claimed in claim 11, characterized in that the device further comprises:
a memory apparatus configured to store one or more algorithms, wherein when the one or more algorithms are implemented by the processor, the processor implements the method.

14. A computer readable memory medium, which stores an algorithm, characterized in that the algorithm realizes the method as claimed in claim 1 when implemented by a processor.
